SEQUENCE LISTING (16 sequences)

SEQ ID NO: 1 - DNA, 22 bp, Artificial sequence
  Description of the artificial sequence outer KDR sense primer
gtcaagggaaagactacgttgg

SEQ ID NO: 2 - DNA, 19 bp, Artificial sequence
  Description of the artificial sequence outer KDR sense primer
agcagtccagcatggtctg

SEQ ID NO: 3 - DNA, 20 bp, Artificial sequence
  Description of the artificial sequence inner KDR sense primer
cagcttccaagtggctaagg

SEQ ID NO: 4 - DNA, 20 bp, Artificial sequence
  Description of the artificial sequence inner KDR sense primer
tcaaaaattgtttctggggc

SEQ ID NO: 5 - DNA, 18 bp, Artificial sequence
  Description of the artificial sequence outer Tie-2/Tek sense primer
tggacctgtgagacgttc

SEQ ID NO: 6 - DNA, 22 bp, Artificial sequence
  Description of the artificial sequence outer Tie-2/Tek antisense primer
ctctaaatttgacctggcaacc

SEQ ID NO: 7 - DNA, 19 bp, Artificial sequence
  Description of the artificial sequence inner Tie-2/Tek sense primer
aggccaacagcacagtcag

SEQ ID NO: 8 - DNA, 22 bp, Artificial sequence
  Description of the artificial sequence inner Tie-2/Tek antisense primer
gaatgtcactaagggtccaagg

SEQ ID NO: 9 - DNA, 21 bp, Artificial sequence
  Description of the artificial sequence outer Ve-cadherin sense primer
tagcattggatactccatccg

SEQ ID NO: 10 - DNA, 20 bp, Artificial sequence
  Description of the artificial sequence outer Ve-cadherin antisense primer
atgaccacgggtaggaagtg

SEQ ID NO: 11 - DNA, 20 bp, Artificial sequence
  Description of the artificial sequence inner VE-cadherin sense primer
ttccgagtcacaaaaaaggg

SEQ ID NO: 12 - DNA, 21 bp, Artificial sequence
  Description of the artificial sequence inner VE-cadherin antisense primer
tatcgtgattatccgtgaggg

SEQ ID NO: 13 - DNA, 23 bp, Artificial sequence
  Description of the artificial sequence outer vWF sense primer
ctgcaaggtcaatgagagagagg

SEQ ID NO: 14 - DNA, 19 bp, Artificial sequence
  Description of the artificial sequence outer vWF antisense primer
gagagcagcaggagcactg

SEQ ID NO: 15 - DNA, 19 bp, Artificial sequence
  Description of the artificial sequence inner vWF sense primer
tgaggagcctgagtgcaac

SEQ ID NO: 16 - DNA, 20 bp, Artificial sequence
  Description of the artificial sequence inner vWF antisense primer
tggagtacatggctttgctg

(19) United States
(12) Patent Application Publication
(10) Pub. No.: US 2006/0051330 A1
(43) Pub. Date: Mar. 9, 2006

(54) Method for carrying out the ex vivo expansion and ex vivo differentiation of multipotent stem cells

(75) Inventors: Dieter Kurt Hossfeld, Hamburg (DE); Walter Fielder, Hamburg (DE); Ursula Gehling, Hamburg (DE); Sonja Loges, Hamburg (DE)

Correspondence Address: MCDERMOTT, WILL & EMERY, 4370 LA JOLLA VILLAGE DRIVE, SUITE 700, SAN DIEGO, CA, 92122, US

(73) Assignee: Universitatsklinikum Hamburg-Eppendorf, Hamburg (DE)

(21) Appl. No.: 10/497,101

(22) PCT Filed: Nov. 22, 2002
(86) PCT No.: PCT/EP02/13142

§ 371 Date: Mar. 9, 2005

(30) Foreign Application Priority Data
Nov. 30, 2001 (DE) 101 58 680.9

(51) Int. Cl. A61K 48/00; C12N 5/08; C12N 15/87

(52) U.S. Cl. 424/93.21; 435/455; 435/368; 435/370

(57) ABSTRACT

The invention relates to a method for carrying out the expansion of multipotent stem cells ex vivo. Moreover, the invention relates to a two-stage method for carrying out the expansion and differentiation of multipotent stem cells ex vivo, in which it is possible for the stem cells to be gene transfected during the first stage, i.e. during the expansion phase. In this phase, the differentiation of the multipotent stem cells takes place optionally in cells of the hematopoietic, endothelial or mesenchymal cell lineage. Stem and progenitor cells as well as mature cells of the hematopoietic, endothelial and mesenchymal cell lineage, which are obtained in this way, can be used, among other things, for the prophylaxis, diagnosis and therapy of human deseases and for tissue engineering.

The invention relates to a method for carrying out the expansion of multipotent stem cells ex vivo. Moreover, the invention relates to a two-stage method for carrying out the expansion and differentiation of multipotent stem cells ex vivo, in which it is possible for the stem cells to be gene transfected during the first stage, i.e. during the expansion phase. In the second phase, the differentiation of the multipotent stem cells takes place optionally in cells of the hematopoietic, endothelial or mesenchymal cell lineage. Stem and progenitor cells as well as mature cells of the hematopoietic, endothelial and mesenchymal cell lineage, which are obtained in this way can be used, among other things, for the prophylaxis, diagnosis and therapy of human diseases and for tissue engineering.

### BACKGROUND AND STATE OF THE ART

a) Endothelial Cell Lineage

The establishment and maintenance of a vessel supply are an absolute precondition for the growth of normal and malignant tissue. Two processes are basically responsible for neovascularisation. The first process, namely vasculogenesis, involves the in situ differentiation of hemangioblastoma to form endothelial cells and their subsequent organisation into a primary capillary plexus. (Risau et al., Development 102, 471-478, 1988; Risau et al., Annu. Rev. Cell Dev. Biol. 11, 73-91, 1995). The second process, the so-called angiogenesis, is defined as the formation of new blood vessels by budding of existing blood vessels. Previously, it had been assumed that the process of vasculogenesis takes place only during the early embryonal phase whereas angiogenesis takes place both prenatally and postnatally. The hemangioblast as a joint stem cell for hematopoietic cells and endothelial cells has recently been identified as a transient cell stage which is detectable for a brief period only during the early embryo development. Subsequently, the hemangioblastoma seems to differentiate out without renewing itself (Choi et al., Development 125, 725-732, 1998). However, it must be pointed out that these results are based on animal experiment studies and cannot necessarily be transferred to the human system. It would be conceivable that, in man, hemangioblastoma or other subpopulations of endothelial progenitor cells persist beyond the embryonal phase and could, in the adult organism, circulate, differentiate and participate in the formation of new blood vessels. This hypothesis has meanwhile been supported by numerous studies (Asahara et al., Science 275, 964-967, 1997; Yin et al., Blood 90, 5002-5012, 1997; Shi et al., Blood 92, 362-367, 1998; Takahashi et al., Nature Med. 5, 434-438, 1999; Asahara et al., Circul. Res. 85, 221-228, 1999; Lin et al., J. Clin. Invest. 105, 71-77, 2000; Kalka et al., Circul. Res. 86, 1198-1202, 2000; Kalka et al., Ann. Thorac. Surg. 70, 829-834, 2000; Bhattacharya et al., Blood 95, 581-585, 2000; Crosby et al., Circul. Res. 87, 728-730, 2000; Murohara et al., J. Clin. Invest. 105, 1527-1536, 2000; Peichev et al., Blood 95, 952-958, 2000).

It has been possible to show that the population of adult human AC133-positive stem and progenitor cells contains endothelial progenitor cells (EPC) which, in the presence of SCGF, a hematopoietic stem cell growth factor, and VEGF, an angiogenic cytokine, differentiate to form functional intact endothelial cells which are capable of forming blood vessels in vivo (Gehling et al., Blood 95, 3106-3112, 2000).

In vitro, the rate of proliferation of endothelial cells is normally very low. Endothelial progenitor cells, too, exhibit only a low growth tendency in conventional cell culture media. The cell counts such as they would be required for many clinical applications could not be achieved in this way. Culture conditions allowing an ex vivo expansion of endothelial progenitor cells and endothelial cells have so far not been developed. Not even with the culture conditions selected in the above-mentioned study (Gehling et al., loc. cit.) has it been possible to induce proliferation of the endothelial progenitor cells in the sense of an expansion. It has merely been possible to achieve a maximum 8-fold multiplication of these progenitor cells. In order to obtain the cell counts of endothelial progenitor cells necessary for clinical applications, however, a hundred-fold expansion needs to be aimed at.

Quirici et al. (Br. J. Heamatol. 115, 186-194, 2001) has described a process for the expansion of endothelial cells in which process CD133-positive bone marrow cells were cultivated initially for 3-4 weeks in the presence of VEGF, bFGF and IGF-1. After purification using Ulex europaeus agglutinin-1 (UEA-1), a further cultivation phase (3-4 weeks) with VEGF, bFGF and IGF-1 follows. Although an approximately 2300 fold expansion is described, it should be noted that the cells purified by UEA-1 represent only approximately 0.5% of the original cells as a result of which the process is practicable only in extremely limited cases, particularly also because of the cultivation phases of a total of 6-8 weeks which is thus very long.

Against this background, the need for an expansion process persists which—preferably starting out from multipotent stem cells obtained by taking blood—provides a sufficient expansion rate and can be carried out without major technical or time expenditure. By providing such a process, numerous new applications would be opened up which have not been feasible previously because the cell material required for this purpose could not be provided at all or in insufficient quantities.

Thus the possible fields of application of endothelial progenitor cells (EPC) and endothelial cells (EC) expanded ex vivo are numerous. In this respect, diagnosis, prophylaxis and therapy of cardiovascular and malignant (such as e.g. neoplastic) diseases and tissue engineering can be mentioned, on the one hand. An example in the field of cardiology is the direct introduction of EPC in underperfused vascular areas of the heart in order to induce the formation of new blood vessels. This method could be transferred to perfusion problems in other organs and areas of the body. In the area of tissue engineering, EPC can be used to produce new blood vessels in vitro for clinical purposes. Moreover, the EPC can serve the purpose of facilitating the supply of vessels in the case of skin transplants and artificially produced (tissue engineered) organs such as liver and pancreas. A further field of application is the use of gene transfected EPC as a vehicle for certain gene products. These genetically modified EPCs can be introduced into the vessels of diseased organs and tumours both for diagnostic and for therapeutic purposes.

Consequently, it is a task of the present invention to provide an expansion method which—preferably starting out from multipotent stem cells obtained by taking blood—provides a sufficient expansion rate and can be carried out without major technical or time expenditure.

b) Hematopoietic Cell Lineage

Since the beginning of the nineties, patients suffering from a malignant tumour have been treated on an increasing scale with high dosage chemotherapy. Since the main side effect consists of an enduring bone marrow aplasia, this therapy is carried out in combination with an autologous transplant of hematopoietic progenitor cells. To obtain a sufficient number of hematopoietic progenitor cells, is necessary to either carry out a large volume removal of bone marrow under full anaesthesy or to carry out one to three leukaphereses.

It is possible for the stem cell reserve of a patient to be insufficient to obtain the quantity of progenitor cells necessary for transplantion from the bone marrow or the peripheral blood. In the last ten years, numerous working groups have worked on the development of culture conditions allowing an ex vivo expansion of hematopoietic progenitor cells (Berenson et al., Blood 77, 1717-1722, 1991; Brandt et al., Blood 79, 634-641, 1992; Haylock et al., Blood 80, 1405-1412, 1992; Brugger et al., Blood 81, 2579-2584, 1993; Sato et al., Blood 82, 3600-3609, 1993; Rice et al., Exp. Hematol. 23, 303-308, 1995; Alcorn et al., J. Clin. Oncol. 14, 1839-1847, 1996; Peters et al., Blood 87, 30-37; 1996, Yonemura et al., Blood 89, 1915-1921, 1997; Reiffers et al., Lancet 354, 1092-1093, 1999, McNiece et al., Blood 96, 3001-3007; 2000). By means of an expansion culture of the progenitor cells preceding the transplantation, it would be possible to reduce the quantity of bone marrow aspirate or leukapheresate to a minimum. For patients with a limited stem cell reserve, a satisfactory progenitor cell transplant could be produced. Moreover, it is hoped that the transplantation of progenitor cells expanded ex vivo would provide a more rapid reconstitution of hematopoeisis.

So far, however, none of the working groups mentioned above has been able to present culture conditions which cause an ex vivo expansion of the “true” hematopoietic stem cell. Instead, the culture conditions developed so far lead to an early differentiation of the stem cells and consequently to an ex vivo expansion of determined progenitor cells, which have lost their stem cell characteristics. These cells are not suitable for transplanting since they are not able to regenerate a permanent hematopoiesis following myeloablative chemotherapy (Shih et al., J. Hematother. Stem Cell Res. 9, 621-628, 2000, McNiece et al., Exp. Hematol. 29, 3-11, 2001).

A further task of the present invention consequently consists of developing a process whose culture conditions permit an ex vivo expansion of transplantable hematopoietic stem cells.

c) Mesenchymal Cell Lineage

In the area of the mesenchymal cell lineage, Reyes et al. (Blood 96, 2615-2625, 2001) has described an ex vivo expansion method in which CD45/glycophorin-A-negative mononuclear bone marrow cells are cultivated in the presence of EGF and PDGF-BB. However, a disadvantage of this process—as in the process described already by Quirici et al. (compare above) for the endothelial cell lineage—consists in that the mononuclear bone marrow cells used represent only a portion of 0.1 to 0.5% of the bone marrow cells. Consequently, this entails additional purification and enrichment stages. Moreover, it must be considered that the CD45⁻/GlyA⁻ cells exhibit only a very low proliferation rate. Thus, the cell reduplication rate is 46-60 hours. Starting out from a removal of 100 ml of bone marrow, 1×10⁸ mononuclear blood cells are obtained of which 0.1-0.5% are CD45⁻/GlyA⁻, i.e. 1-5×10⁵ CD45⁻/GlyA⁻cells. After 14 days in the culture, only 6.4×10⁶ to 3.2×10⁷ stem cells are thus obtained. For this reason, the expansion method according to Reyes et al. is of only little practical importance, in particular for clinical use.

The Present Invention:

The task of the present invention consists of avoiding the disadvantages known from the state of the art and of providing an expansion method by means of which markedly higher cell counts can be achieved during expansion than has been possible so far in the state of the art. In particular, a method is to be provided by means of which it is possible to multiply, in a controlled manner, progenitor cells and mature cells of different cell lineages (hematopoietic, endothelial and mesenchymal cell lineages) equally in different differentiation stages. The method should be practicable without any major technical or time expenditure and be preferably based on multipotent stem cells accessible by the simple taking of blood.

According to the invention, the task is achieved by way of methods for carrying out the expansion of multipotent stem cells in which multipotent stem cells are cultivated in the presence of Flt3 ligand and at least one growth factor from the group consisting of SCF, SCGF, VEGF, bFGF, insulin, NGF and TGF-β. In each case, IGF-1 and/or EGF can optionally additionally be used.

According to a particular embodiment, one of the following combinations is chosen:
a) Flt3 ligand and VEGF,
b) Flt3 ligand, SCGF and VEGF,
c) Flt3 ligand and EGF,
d) Flt3 ligand, EGF and bFGF,
e) the growth factors mentioned in a) to d) in combination with IGF-1 and/or EGF.

Surprisingly enough, it is possible by using the above-mentioned growth factors to achieve a more than hundred fold multiplication of the cell counts used to obtain, starting out, for example, from only 50 ml of leukapheresis product 1×10⁹ to 1×10¹⁰ multipotent stem cells already after a 14 day culture. The expansion can thus be carried out on a markedly larger scale than in the state of the art. A further advantage, consists of the fact that it is possible to use sources for stem cells which, such as e.g. blood, are obtainable in a simple manner. Unexpectedly, the use of Flt3 ligand, which is a hematopoietic growth factor, in combination with the above-mentioned growth factors do not lead to a premature differentiation of the stem cells, not even in the direction of the hematopoietic cell lineage. This has the particular advantage that it is possible to allow the multipotent stem cells to mature after expansion in a subsequent differentiation phase. At the same time, the separation of expansion and differentiation according to the invention makes it possible, in an advantageous manner, to effect a genetically engineered modification of the still multipotent stem cells. In other words, it is possible to transfect the stem cells, while they are strongly proliferating, with vectors which preferably contain nucleic acid sequences encoding proteins or polypeptides which are not naturally expressed in these cells.

Since it has come to light that the use of Flt3 ligand promotes differentiation in the presence of VEGF and bFGF, this combination should be avoided if an expansion of multipotent stem cells is to be aimed at exclusively, i.e. without or without significant differentiation. Instead, it is possible according to the invention for the differentiation of the expanded multipotent stem cells to take place in the subsequent second step by means of which a differentiation can be carried out in a targeted manner into one of the three cell lineages (endothelial, hematopoietic and mesenchymal).

Consequently, the subject matter of the invention also consists of a two-phase method (two-phase culture system) in which multipotent stem cells are expanded and developed to produce human progenitor cells and mature cells of the hematopoietic, endothelial and mesenchymal cell lineage. The above-mentioned expansion process according to the invention corresponds to phase I of the two-phase method. Consequently, for simplification, reference will be made in the following to phase I, the details given also applying equally to the expansion method (i.e. without subsequent differentiation phase).

Consequently, the invention also relates to a method for carrying out in vitro (ex vivo) expansion and differentiation of multipotent stem cells in which
a) the expansion process according to the invention is carried out in a first phase for the expansion of multipotent stem cells (i.e. in the presence of Flt3 ligand and at least one growth factor from the group consisting of SCF, SCGF, VEGF, bFGF, insulin, NGF and TGF-β (if necessary in combination with IGF-1 and/or EGF in each case) and
b) the expanded cells are differentiated in a second phase, the cells being
   (i) cultivated for (the induction of) hematopoietic differentiation in the presence of at least one growth factor from the group consisting of G-CSF, GM-CSF, M-CSF, IL-3, IL-6, IL-11, TPO and EPO, optionally in combination with at least one growth factor from the group consisting of IL-1, SCF and SCGF,
   (ii) cultivated for (the induction of) endothelial differentiation in the presence of at least one growth factor from the group consisting of VEGF, aFGF, bFGF, ECGS, AP-1, AP-2, NGF, CEACAM, pleiotrophin, angiogenin, PlGF, and HGF, optionally in combination with at least one growth factor from the group consisting of LIF, EGF, IGF-1, PDGF, PDECGF, TGFα, TGFβ, TNFα, estrogen, proliferin, IL-3, G-CSF, GM-CSF, EPO SCF and SCGF,
   (iii) cultivated for (the induction of) mesenchymal differentiation in the presence of at least one growth factor from the group consisting of PDGF-BB, TGF-β and BMP-4, optionally in combination with at least one growth factor from the group consisting of EGF, aFGF, bFGF, IGF-1, SCF and SCGF,
   (iv) cultivated for (the induction of) neuronal differentiation in the presence of at least one growth factor from the group consisting of NGF, CNTF, GDNF and BDNF, optionally in combination with at least one growth factor from the group consisting of EGF, bFGF, IGF-1, IL-1b, Il-6, Il-11, LIF, Flt3 ligand, SCF and BMP-4, or
   (v) cultivated for (the induction of) hepatocytic differentiation in the presence of HGF, optionally in combination with at least one growth factor from the group consisting of EGF, IGF-1, insulin, HCG, KGF, TNF-α, Flt3 ligand, SCF and SCGF.

To carry out the expansion (in line with the first phase of the two-stage method), the use of Flt3 ligand in the following combination is preferred:
a) Flt3 ligand and VEGF,
b) Flt3 ligand, SCGF and VEGF,
c) Flt3 ligand and EGF,
d) Flt3 ligand, EGF and bFGF,
e) the growth factors mentioned in a) to d) in combination with IGF-1 and/or EGF.

In the second phase, use is made according to a particular embodiment
(i) of a combination of SCF, IL-3, IL-6, G-CSF, GM-CSF and EPO for the hematopoietic differentiation
(ii) of a combination of SCGF and VEGF for the endothelial differentiation
(iii) of a combination of EGF, PDGF-BB, IGF-1, bFGF and BMP-4 for the mesenchymal differentiation
(iv) of a combination of BDNF, GDNF, EGF and bFGF for the neuronal differentiation or
(v) of a combination of Flt3 ligand, SCF, HGF and TGF-β for the hepatocytic differentiation.

In a particularly easy manner, the method can additionally be used for a gene transfection of the stem cells without impeding cell expansion. The gene transfected stem cells can be differentiated into the hematopoietic, endothelial and mesenchymal cell lineage in a manner analogous to the genetically non-modified stem cells.

During gene transfection, a nucleic acid sequence that codes for a protein or polypeptide not naturally expressed in the cells (in the following referred to as “foreign gene”) is introduced.

The multipotent stem cells can be obtained from mobilised or non-mobilised autologous peripheral blood or bone marrow of the patient or from the blood from the veins of the umbilical cord. The mobilisation therapy can consist of a subcutaneous or intravenous injection of growth factors such as G-CSF, GM-CSF or SCF and/or intravenous or oral application of cytostatics. Obtaining the multipotent stem cells from G-CSF mobilised peripheral blood (freshly obtained or frozen leukapheresis products) represents a particular embodiment of the invention. The multipotent stem cells can be obtained in the mononuclear cell fraction. By using antibodies which recognise special antigens to multipotent stem cells, it is possible to isolate the multipotent stem cells. The following antibodies can be used: anti-CD7 mAb, anti-CD31 mAb (PECAM-1), anti-CD34 mAb, anti-CD54 (ICAM-1) mAb, anti-CD90 (Thy-1) mAb, anti-CD114 (G-CSF-R) mAb, anti-CD116 (GM-CSF-R) mAb, anti-CD117 (c-kit) mAb, anti-CDw123 (IL-3R α chain) mAb, anti-CD127 (IL-7R) mAb, anti-AC133 mAb, anti-CD135 (Flk3/Flk2) mAb, anti-CD140b (PDGF-Rβ) mAb, anti-CD144 (VE-cadherin) mAb, anti-CD164 mAb, anti-CD172a mAb, anti-CD173 mAb, anti-CD174 mAb, anti-CD175 mAb, anti-CD176 mAb, anti-CD184 (CXCR4) mAb, anti-CD201 (endothelial cell protein C receptor) mAb, anti-CD202b (Tie-2/Tek) mAb, anti-CD224 mAb, anti-CD227 (MUC-1) mAb, anti-CD228 mAb, anti-CD243 (MDR-1) mAb, anti-EGF-R mAb, anti-FGF-R mAb, anti-P1H12 mAb, anti-KDR mAb, anti-EN4 mAb, anti-BENE mAb. As an alternative to antibodies, lectins such as e.g. Ulex europaeus agglutinin-1 can be used for the selection of the multipotent stem cells. In addition, the multipotent stem cells can also be obtained by depletion. For this purpose, mAb CD45 can be used. In principle, the multipotent stem cells can be obtained in the following cell populations: AC133⁺ CD34⁺, AC133⁺ CD34⁻, AC133⁻ CD34⁻. The selection of the overall population of AC133-positive stem cells and progenitor cells is recommended.

The individual phases of the culture system according to the invention will be explained in further detail in the following:

Phase I of the Culture System: Expansion Phase

After obtaining the multipotent stem cells, these cells are expanded ex vivo in suspension cultures. IMDM, MEM, DMEM, X-Vivo10, RPMI, M-199 medium, EGM-2 can be used as basal medium. The basal medium can be supplemented with fetal calf serum, horse serum or human serum. As an alternative, the multipotent stem cells can be expanded serum-free. For the expansion phase, the above-mentioned (preferably recombinant) human growth factors an be used. Moreover, the medium can be supplemented with hydrocortisone. The ex vivo expansion of the multipotent stem cells in IMDM+10% FBS+10% horse serum+10⁻⁶ mol/l hydrocortisone+SCGF+Flt3 ligand+VEGF represents a preferred embodiment according to the invention. SCGF can be replaced by SCF without problem.

During the expansion phase, genetic material can be transferred to the multipotent stem cells. The genetic material which is transferred to the multipotent stem cells expanded ex vivo can be genes which encode numerous proteins. These genes comprise those encoding fluorescent proteins such as e.g. GFP. Moreover, these genes also comprise those encoding different hormones, growth factors, enzymes, cytokines, receptors and antitumour substances. In addition, the genes can encode a product which controls the expression of another gene product or genes which block one or several steps of a biological reaction sequence. In addition, the genes can encode a toxin which is fused with a polypeptide, e.g. a receptor ligand, or with an antibody which binds the toxin to the target cell. Correspondingly, the gene can encode a therapeutic protein which is fused with a “targeting” polypeptide in order to transfer a therapeutic effect onto a diseased organ or tissue.

The nucleic acids are introduced into the multipotent stem cells expanded ex vivo by means of a method which guarantees their incorporation and expression in the stem cells. These methods can comprise vectors, liposomes, naked DNA, electroporation etc.

Phase II of the Culture System: Differentiation Phase

In suspension cultures, the multipotent stem cells can be differentiated directly after isolation or after prior ex vivo expansion, genetically natively or in a modified manner into the hematopoietic, endothelial or mesenchymal cell lineage. The following media can be used as basal medium: IMDM, MEM, RPMI, M-199, X-Vivo10, EGM-2, Williams medium E, SATO medium, DMEM or DMEM-F12. The basal medium can be supplemented with fetal calf serum, horse serum or human serum. As an alternative, serum-free culture conditions can be used.

In order to induce a hematopoietic differentiation, the following (preferably recombinant) human growth factors are added: G-CSF, GM-CSF, M-CSF, IL-3, IL-6, IL-11, TPO and/or EPO. Additionally, one or several of the following (preferably recombinant) human growth factors can be used: IL-1, SCF and SCGF. The use of SCF, IL-3, IL-6, G-CSF and TPO in combination with EPO represents a particularly preferred embodiment of the invention.

The induction of the differentiation of the multipotent stem cells into the endothelial cell lineage, i.e. into endothelial progenitor cells and into mature endothelial cells is achieved by using the following (preferably recombinant) human growth factors: VEGF, bFGF and/or ECGS. Additionally, one or several of the following (preferably recombinant) human growth factors can be used: AP-1, AP-2, LIF, EGF, IGF-1, NGF, CEACAM, HGF, SCF and SCGF. The use of SCF, VEGF, bFGF, IGF-1, EGF, LIF plus AP-1 represents an embodiment preferred according to the invention.

In order to induce mesenchymal differentiation, the following (preferably recombinant) human growth factors are added: PDGF-BB, TGF-β and/or BMP-4. Additionally, one or several of the following (preferably recombinant) human growth factors can be used: EGF, aFGF, bFGF, IGF-1, SCF and SCGF. The use of EGF, PDGF-BB, IGF-1 and bFGF in combination with BMP-4 represents a particularly preferred embodiment of the invention.

The induction of the differentiation of the multipotent stem cells into the neuronal cell lineage, i.e. into neuronal progenitor cells and into mature neuronal cells is achieved by using the following (preferably recombinant) human growth factors: NGF, CNTF, GDNF and/or BDNF. Additionally one or several of the following (preferably recombinant) human growth factor can be used: EGF, bFGF, IGF-1, IL-1b, Il-6, Il-11, LIF, Flt3 ligand, SCF and BMP-4. The use of BDNF, GDNF, EGF plus bFGF represents an embodiment preferred according to the invention.

In order to induce a hepatocytic differentiation, the (preferably recombinant) human growth factor HGF is added. Additionally, one or several of the following (preferably recombinant) human growth factors can be used: EGF, IGF-1, insulin, HCG, KGF, TNF-α, Flt3 ligand, SCF and SCGF. The use of Flt3 ligand, SCF, HGF plus TGF-β represents a particularly preferred embodiment of the invention.

In order to characterise the development stage of the cells in the culture, it is necessary to check the differentiation phase lasting approximately 10 to 14 days at regular intervals. A functional examination of the cells in the culture, e.g. in the form of a colony assay, is suitable, for example. During the differentiation of the multipotent stem cells into the endothelial cell lineage, the EPCs loose the ability to form blood cell colonies, e.g. with an increasing differentiation. By removing cell samples in phase II, it is thus possible to check at regular intervals of e.g. 1 to 3 days whether and to what extent the ability of the cells to form colonies of the cell lineage not desired in each case has changed. As soon as the cells form only colonies of the desired cell lineage, the differentiation phase has reached the stage in which only progenitor cells of this cell lineage are present. The cells can either be removed and/or isolated for further applications or differentiated into mature cells of the desired cell lineage.

In addition to or instead of the colony assay, the cells can be examined in phase II by means of immunocytochemistry in order to verify the formation of certain surface structures on the cells during the differentiation phase. The results of the functional assay can be compared in an advantageous manner with those of the immunocytochemical analyses in order to find out which surface structures need to be formed if progenitor cells of the desired cell lineage are present, i.e. the cells have not yet matured but nevertheless lost the ability of the other cell lineages to form colonies.

The progenitor cells isolated in the manner described above must be used either immediately in the desired manner, i.e. for the planned application, or be frozen. For endothelial progenitor cells, a medium consisting of DMSO, IMDM and HSA (preferably 40% IMDM+50% HSA+10% DMSO) has proved advantageous.

The present invention allows the use of ex vivo expanded multipotent stem cells and of hematopoietic, endothelial and mesenchymal progenitor cells and mature cells for the diagnosis, prophylaxis and therapy of cardiovascular and malignant diseases. In addition, the multipotent stem cells, endothelial progenitor cells and mature endothelial cells expanded ex vivo can be used for coating surfaces. The multipotent stem cells expanded ex vivo and the endothelial and mesenchymal progenitor cells and mature cells can also be used for tissue engineering of organs and tissues.

In the following, a few practical examples of cells expanded and differentiated in vitro are provided as examples, the uses mentioned having the purpose merely of clarifying the application possibilities for the multipotent stem cells according to the invention during expansion and/or differentiation, without restricting the invention thereto.

Application I: Transplantation of Multipotent Stem Cells for Hematopoietic Differentiation In Vivo

The multipotent stem cells expanded ex vivo can be used for allogenic or autologous transplantation in patients who, due to a malignant disease, are treated by myeloablative chemotherapy in order to regenerate blood formation. In this case, the growth factor G-CSF is first administered to the patients in order to effect a mobilisation of the bone marrow stem cells into the peripheral blood. Instead of leukaphereses, blood can be taken from the patients in the normal manner. From the peripheral blood, the stem cells are then isolated and the quantity of stem cells required for a transplant generated by ex vivo expansion. It is thus possible to avoid subjecting the patients to stresses and risks connected with the execution of leukaphereses. On the one hand, the transplant may consist exclusively of multipotent stem cells expanded ex vivo. Alternatively, a transplant can be used which consists of expanded stem cells and endothelial progenitor cells. By the additional use of the endothelial progenitor cells, the reconstitution of the bone marrow function of the patients can be accelerated.

Application II: Transplantation of Genetically Modified Stem Cells and Progenitor Cells

Since the expansion method according to the invention and/or phase I of the two-stage method is a phase in which the multipotent stem cells proliferate strongly, a simultaneous gene transfection can be carried out advantageously. Corresponding methods for gene transfection by vectors, liposomes, naked DNA or electroporation are well known to the person skilled in the art (compare “References”). In this connection, it may be advantageous to use a vector for gene transfection which allows an expression of the foreign gene inducible externally, i.e. of the gene not naturally expressed in the cells, such as e.g. the so called “Tet-on-system”, consisting of a transactivator construct and a responder construct (compare Gossen M., Bujard H. Proc. Natl. Acad. Sci. 89, 5547-5551, 1992; Gossen et al., Science 268, 1766-1769, 1995; Puttin et al., Am. J. Physiol. Renal Physiol. 281, F1164-72, 2001).

The stem cells expanded ex vivo and the endothelial progenitor cells can thus be genetically modified before transplantation and used for diagnostic and therapeutic applications in the case of malignant tumours and leukaemia. It is, for example, possible to modify the multipotent stem cells and endothelial progenitor cells expanded ex vivo genetically in such a way that they inhibit angiogenesis. This can be achieved e.g. by introducing a gene which encodes an angiogenesis inhibiting substance. The angiogenesis inhibiting substances comprise e.g. endostatin or angiostatin as well as antibodies or antisense nucleic acids against angiogenic cytokines, such as e.g. VEGF. Another possible application is gene therapy of hereditary diseases such as for example hemophilia A and B (compare Mannuci P M, Tuddenham E G. N. Engl. J. Med. 344, 1773-1779, 2001; Emilien et al., Clin. Lab. Hematol. 22, 313-322, 2000), Gaucher's disease (compare Barranger et al., Baillieres Clin. Hematol. 10, 765-768, 1997), glycogenosis (type I-III) (compare Elpeleg O N. J. Pediatr. Endocrinol. Metab. 12, 363-379, 1999), mucopolysaccharidosis (type I-VII) (compare Caillaud C, Poenaru L. Biomed. Pharmacother. 54, 505-512, 2000), Niemann-Pick disease (compare Millat et al., Am. J. Hum. Genet. 69, 1013-1021, 2001; Miranda et al., Gene Ther. 7, 1768-1776, 2001), Hirschsprung's disease (compare Amiel et al., J. Med. Genet. 38, 729-739, 2001), Fanconi anemia (compare Yamashita T. Int. J. Hematol. 74, 33-41, 2001), Chediak-Higashi syndrome (compare Ward et al., Traffic 1, 816-822, 2000), thalassemia (compare Weatherhall D J. Nat. Rev. Genet. 2, 245-255, 2001), sickle cell anaemia (compare Chui D H, Dover G J. Curr. Opin. Pediatr. 13, 22-27, 2001) etc.

Methods for gene transfection suitable within the framework of the present invention are described in the publications quoted in the “References” section, these processes being mentioned only as examples without being limited to these methods.

Application III: Diagnosis of Metastases and Ischemic Diseases

III a.) Diagnosis of Metastases

As a special application in oncology, the ex vivo-expanded multipotent stem cells and/or the endothelial progenitor cells can be radioactively labelled with 18F fluorodeoxyglucose (¹⁸F-FDG) or with ¹¹¹indium and administered to patients intravenously in order to visualise metastases. The administered cells are built up in the tumour tissue (compare de Bont et al., Cancer Research 61, 7654-7659, 2001), as a result of which the metastases can be visualised by diagnostic routine processes such as positron emission tomography (PET, for the detection of ¹⁸F-FDG labelled cells) and/or simple scintigraphy (for the detection of ¹¹¹indium-labelled cells).

III b.) Diagnosis of Ischemic Lesions

The radioactive labelling of ex vivo-expanded multipotent stem cells and/or the endothelial progenitor cells with 18F-fluorodeoxyglucose (¹⁸F-FDG) or with ¹¹¹indium can also be used for the diagnosis of ischemic diseases. Following intravenous administration, the labelled cells migrate via the circulation into ischemic regions of the organism in order to participate there in angioneoplasm (compare survey by Masuda et al., Hum. Cell 13, 153-160, 2000). In this way, it is possible to detect also undervascularizations which are clinically without symptoms. The visualisation of the labelled cells takes place by PET and/or scintigraphy analogous to the above-mentioned process.

Application IV: Angioneoplasm In Vivo

The ex vivo-expanded multipotent stem cells and the endothelial progenitor cells and mature endothelial cells can also be used for the therapy of diseases involving a reduced vascular supply. It is, for example, possible to introduce the ex vivo-expanded multipotent stem cells, the endothelial progenitor cells or the mature endothelial cells directly into an organ and/or vessel system in order to induce the formation of new blood vessels therein. The reduced vascular supply can be due to an ischemic disease or an autoimmune disease. Affected tissues can comprise muscles, the brain, kidneys, lung. The ischemic tissues can consist particularly of a myocardial ischemia, ischemic myocardiopathy, renal ischemia, pulmonal ischemia or an ischemia of the extremities. The ex vivo-expanded stem cells and the endothelial progenitor cells can be genetically modified before introduction into the diseased organ and/or vessel in order to increase the therapeutic effect. It is, for example, possible to transfect the ex vivo-expanded stem cells and endothelial progenitor cells with a gene which encodes a vasodilatory substance.

Application V: Reendothelialisation of Vessels

As a special application in cardiology, both the ex vivo-expanded stem cells and the endothelial progenitor cells and mature endothelial cells can be used for the treatment of diseases and injuries of the coronary arteries. It is, for example, possible to apply the multipotent stem cells or the endothelial progenitor cells following an angioplasty or rotablation directly intracoronal in order to accelerate reendothelialisation of the injured coronary sections, thus preventing restenosis. This application can also be transferred to the treatment of diseases and injuries of arteries in other localities such as e.g. vessels of the extremities, by injecting the expanded stem cells, the endothelial progenitor cells or the mature endothelial cells directly into the vessel concerned.

Application VI: Coating of Coronary Stents

Moreover, the endothelial progenitor cells and mature endothelial cells obtained by the differentiation of the multipotent stem cells can be used for coating coronary stents which are implanted following angioplasty or rotablation in order to prevent restenosis. In this case, the endothelial progenitor cells or the mature endothelial cells can be applied either directly onto the stent surface or on matrix-coated stents.

Different stent surfaces can be used: ceramics, PTFE, gold, titanium etc. The matrix can consist e.g. of fibronectin, collagen, heparin, gelatine, fibrin, silicone, phosphoryl choline or matrigel. Additionally, the matrix can be coupled with antibodies binding endothelial cell-specific or progenitor cell-specific surface antigens. The following antibodies can be used: anti-CD7 mAb, anti-CD31-mAb, anti-CD34 mAb, anti-CD54 (ICAM-1) mAb, anti-CD62e mAb (E-selection), anti-CD90 (Thy-1) mAb, anti-CD106 mAb (VCAM-1), anti-CD114 (G-CSF-R) mAb, anti-CD116 (GM-CSF-R) mAb, anti-CD117 (c-kit) mAb, anti-CDw123 (IL-3R α chain) mAb, anti-CD127 (IL-7R) mAb, anti-AC133 mAb, anti-CD135 (Flk3/Flk2) mAb, anti-CD140b (PDGF-RP) mAb, anti-CD144 (VE-cadherin) mAb, anti-CD164 mAb, anti-CD172a mAb, anti-CD173 mAb, anti-CD174 mAb, anti-CD175 mAb, anti-CD176 mAb, anti-CD184 (CXCR4) mAb, anti-CD201 (endothelial cell protein C receptor) mAb, anti-CD202b (Tie-2/Tek) mAb, anti-CD224 mAb, anti-CD227 (MUC-1) mAb, anti-CD228 mAb, anti-CD243 (MDR-1) mAb, anti-EGF-R mAb, anti-FGF-R mAb, anti-P1H12 mAb, anti-KDR mAb, anti-BENE mAb antibodies against lectins. The endothelial progenitor cells can be used for the coating in a genetically unmodified or gene transfected state. For the transfection, genes encoding a vasodilatory substance such as e.g. NO synthase or genes encoding an antithrombotic substance such as e.g. antithrombin III can be used.

Application VII: Coating of Vascular Valves

A further use of the endothelial progenitor cells and mature endothelial cells obtained in culture consists of the coating of biomechanical vascular valves of the heart in order to prevent thrombosation of implanted vascular valves.

According to the two above-mentioned practical examples, the invention also relates to methods for coating implantable materials, in particular coronary stents and vascular valves, in the case of which the two-stage expansion/differentiation method according to the invention is carried out and, in the case of endothelial differentiation during phase II and/or at the end of phase II (depending on whether a coating with EPCs and/or mature ECs is desired), the material to be implanted, which is preferably coated with fibronectin, is transferred into the culture medium in which the differentiation of the cells takes place. According to a preferred embodiment of the invention, the stem cells can be gene transfected in phase I such that coating takes place with gene-transfected EPCs and/or ECs.

Application VIII: Tissue Engineering of Organs

A possible field of application for the ex vivo-expanded multipotent stem cells and for the endothelial and mesenchymal progenitor cells is tissue engineering. The ex vivo-expanded multipotent stem cells can be used in order to produce organ-specific tissues such as brain, liver, heart, cartilage, bone, retinal, muscle or connective tissue in vitro. For this purpose, the stem cells are cultivated in special basal media. To generate neuronal cells, the media SATO medium or DMEM-F12, for example, can be used. For the preparation of liver cells, media such as e.g. Williams medium E can be used. The cultures can contain additions of serum. Alternatively, serum-free culture systems can be used.

For the induction of the neuronal differentiation, the multipotent stem cells can be cultivated in the presence of at least one growth factor from the group consisting of NGF, ciliary neurotrophic factor (CNTF), GDNF and BDNF and optionally in combination with at least one growth factor from the group consisting of EGF, bFGF, IGF-1, IL-1b, IL-6, IL-11, LIF, Flt3 ligand, SCF and SCGF.

For tissue engineering of liver cells, the multipotent stem cells can be cultivated in the presence of HGF and, optionally, in combination with at least one growth factor from the group consisting of EGF, IGF-1, insulin, HCC, keratinocyte growth factor, TNF-α, TGF-β, Flt3 ligand, SCF and SCGF.

In connection with the tissue engineering of organs and tissues, the following methods can be used: liver (compare Torok et al., Dig. Surg. 18, 196-203, 2001), brain (compare Woerly S. Neurosurg. Rev. 23, 59-77, 2000; Tresco P A. Prog. Brain Res. 128, 349-363, 2001), heart (compare Mann B K, West J L. Anat. Rec. 263, 367-371, 2001), cartilage (compare Laurencin et al., Annu. Rev. Biomed. Eng. 1, 19-46, 1999; Lu et al., Clin. Orthop. 391, S251-270; Gao et al., Tissue Eng. 7, 363-371, 2001), bone (compare Doll et al., Crit. Rev. Eukaryot. Gene Expr. 11, 173-198, 2001; Gao et al., Tissue Eng. 7, 363-371, 2001), retina (compare Lu et al., Biomaterials 22, 3345-55, 2001), muscle tissue (compare Polinkovic et al., Crit. Rev. Eukaryot. Gene Expr. 11, 121-129, 2001), connective tissue (compare Pieper et al., Biomaterials 21, 1689-1699, 2001), skin (Houzelstein et al., Development 127, 2155-64, 2000; Ng et al., Tissue Eng. 7, 441-455, 2001), kidney (compare Amiel et al., World J. Urol. 18, 71-79, 2000; Poulson et al., J. Pathol. 195, 229-235, 2001). When establishing the vessel supply in organs prepared by tissue engineering, the method of Kaihara et al. (Tissue Eng. 6, 105-117, 2000) can be used.

To produce artificial tissues, in particular brain, liver, kidney, heart, bone, retinal, muscle or connective tissue or skin, a matrix can be provided which is brought into contact with the multipotent stem cells, progenitor cells and/or differentiated cells expanded according to the invention. This means that this matrix is transferred into a suitable vessel and a layer of the cell-containing culture medium is placed on top (before or during the differentiation of the expanded multipotent stem cells). The term “matrix” should be understood in this connection to mean any suitable carrier material to which the cells are able to attach themselves or adhere in order to form the corresponding cell composite, i.e. the artificial tissue. Preferably, the matrix or carrier material, respectively, is present already in a three-dimensional form desired for later application. According to a particular embodiment of the invention, bovine pericardial tissue is used as matrix which is crosslinked with collagen, decellularised and photofixed (CardioFix™, Sulzer Medica, Forich, Switzerland).

Application IX: Tissue Engineering of Blood Vessels

Moreover, the ex vivo expanded multipotent stem cells and the endothelial progenitor cells and mature endothelial cells can be used for the in vitro preparation of blood vessels. The blood vessels generated in vitro can be implanted as vascular transplants in patients with coronary heart disease or peripheral arterial vascular occlusions and represent an alternative to the bypass operation and to implanting of artificial vessel prostheses.

Regarding the process for the production of artificial blood vessels, reference is made to the details provided regarding the production of artificial tissue, in particular regarding the “matrix” used, in order to avoid repetition. The matrix is preferably preformed in the form of a cylinder.

Application X: Angioneoplasm in Organ Transplants and Tissue Transplants

The ex vivo-expanded multipotent stem cells and the endothelial progenitor cells can also be used to improve or to guarantee the vascular supply for skin transplants. The skin transplants can comprise mesh grafts or skin transplants produced by tissue engineering.

In addition, the ex vivo expanded multipotent stem cells and the endothelial progenitor cells can be used in order to guarantee a vascular supply for organs or tissues produced by tissue engineering. The organs or tissues can comprise e.g. liver, kidney or cartilage. By using autologous multipotent stem cells or autologous endothelial progenitor cells, vascular systems can be produced individually for the patient in order to possibly prevent a host versus transplant reaction (transplant rejection).

With respect to the above-mentioned uses, a further object of the present invention consequently consists of a method for the production of a pharmaceutical composition in which the method according to the invention for the expansion of multipotent stem cells is carried out. Insofar as additionally to or instead of the stem cells, progenitor cells (e.g. endothelial progenitor cells) and/or matured cells (e.g. mature endothelial cells) are to be used, the differentiation phase may follow according to the invention such that the two-stage expansion/differentiation method is carried out for the preparation of the pharmaceutical composition, the cells being isolated during and/or at the end of phase II, depending on the desired degree of differentiation. The cells obtained in each case can be used directly for therapy, preferably by being taken up in 0.9% saline solution or, insofar as required, processed in another way for the administration concerned. If necessary, this includes radioactive labelling of the cells.

According to a particular embodiment of the invention, the pharmaceutical composition may contain a mixture of expanded multipotent stem cells and endothelial progenitor cells. The process for the preparation of the pharmaceutical composition consequently includes, if necessary, the execution of the expansion/differentiation method according to the invention (i.e. phase I and II), wherein cells obtained in phase I are combined with EPCs isolated in phase II.

According to a further embodiment of the invention, the process for the production of a pharmaceutical composition can also include a gene transfection, i.e. the introduction of foreign genes into the multipotent stem cells, the transfection taking place within the framework of the expansion process (e.g. during the two-stage process during the expansion phase, phase I). Insofar as the genetically modified multipotent stem cells are further differentiated, it is also possible to provide a pharmaceutical composition which contains both gene transfected stem cells and gene transfected progenitor cells.

According to the invention, the term “pharmaceutical composition” includes both preparations for therapeutic application and agents for diagnostic purposes.

Moreover, the invention relates to the use of the cells obtained by the expansion process according to the invention and of the cells obtained by the two-stage expansion/differentiation process according to the invention (i.e. multipotent stem cells, progenitor cells and matured cells) for the production of artificial organs and tissues, in particular of brain, liver, kidney, heart, cartilage, bone, retinal, muscle or connective tissue or skin.

The subject matter of the invention moreover consists of pharmaceutical compositions, implantable materials and artificial organs and tissue, in particular including the blood vessels, produced by using the expanded multipotent stem cells, progenitor cells and/or mature cells produced according to the invention (and/or obtainable by using a process according to the invention).

### ADVANTAGES OF THE INVENTION

The present invention describes a culture system which allows an ex vivo expansion of multipotent human stem cells. In comparison with culture systems previously described, the present invention has the advantage that no or no major differentiation of the stem cells occurs during the expansion phase. As a result, the stem cells retain their regenerative capacity and can be used for autologous or allogeneic transplants in patients with malignant diseases. Moreover, they can be used for tissue engineering. The invention is moreover characterised in that the multipotent stem cells can be gene transfected under the culture conditions developed. As a result, new approaches for the diagnosis and therapy of cardiovascular and malignant diseases are obtained. Finally, the invention makes it possible that endothelial progenitor cells are multiplied a hundred fold in the culture system and consequently cell counts are reached such as they are necessary for clinical applications. In this respect, the culture system has the advantage that both the multipotent stem cells and the endothelial progenitor cells can be produced without major expenditure on equipment.

Important applications of the stem cells expanded and/or differentiated according to the invention are summarised in the following as an example:
Transplantation with ex vivo expanded multipotent stem cells
Transplantation with ex vivo expanded multipotent stem cells+endothelial progenitor cells
Transplantation with genetically modified multipotent stem cells with the following transgene:
   angiogenic inhibiting gene
   angioneoplasm promoting gene
Transplantation with genetically modified multipotent stem cells for the therapy of hereditary diseases
Diagnosis of metastases
Diagnosis of ischemia
Angioneoplasm in vivo
Reendothelialisation of vessels in vivo
Tissue engineering of brain, liver, kidney, heart, cartilage, bone, retinal, muscle or connective tissue or of skin
Production of artificial blood vessels
Vascular supply for skin transplants
Vascular supply for organs or tissue produced artificially (by tissue engineering).

In the following, the invention will be described by way of examples.

### EXAMPLES

The substances, growth factors and antibodies mentioned above and in the following examples are either commercially available or they can be produced and/or obtained according to known methods. A survey of the relevant publications are given in the appendix under “Reference”.

### Example 1

Sample Preparation:

For this example, a leukapheresis product, kept under cryogenic conditions, of a patient was used who, as a result of a malignant disease, had been assigned to high dosage chemotherapy with autologous stem cell transplantation. However, fresh leukapheresis products or G-CSF mobilised, non-pheresised blood can also be processed. The sample kept under cryogenic conditions was defrosted in a first step at 37° C. in a water bath and transferred into a buffer consisting of PBS, 0.5% HSA and 0.6% ACD-A. The sample was then centrifuged for 15 minutes at 900 rpm and 4° C. The cell pellet obtained was resuspended in PBS+5% HSA. Subsequently, DNAse (100 U/ml) was added to this PBS solution and the sample was incubated for 30 minutes on an automatic mixer.

Fresh leukapheresis product and peripheral, non-pheresised blood can be passed directly to density gradient centrifugation.

Immunomagnetic AC133 Selection:

By density gradient centrifugation via Fikoll-Hypaque, the mononuclear cell fraction (MNC) of the leukapheresis product was obtained. For this purpose, the sample was centrifuged for 20 minutes at 2000 rpm and 4° C. Subsequently, the sample was washed twice for 10 minutes at 1200 rpm in PBS+0.5% HSA+DNAse (100 U/ml). The MNC were then resuspended in PBS+0.5% HSA, incubated with AC133-conjugated microbeads (AC133 isolation kit, Miltenyi Biotec, Bergisch-Gladbach) for 30 minutes at 4° C. and washed in PBS+0.5% HSA for 10 minutes at 1200 rpm. The AC133 selection was then carried out on the autoMACS (Miltenyi Biotec; Posseldx software program). Following each selection, the degree of purity was determined by FACS analysis.

Suspension Cultures:

The freshly isolated AC133⁺ cells were cultivated in fibronectin-coated plates with 24 well plates at a cell density of 2×10⁶ cells/ml in IMDM+10% FCS+10% horse serum+10⁻⁶ mole/l hydrocortisone. For the expansion of the AC133⁺ cells, the following recombinant human growth factors were added to the medium: SCGF (100 ng/ml; TEBU, Frankfurt), Flt3 ligand (50 ng/ml; TEBU) and VEGF (50 ng/ml; TEBU) and the cells were incubated for 14 days at 37° C. in 5% CO₂. For the differentiation of the stem cells, the medium was supplemented with SCGF (100 ng/ml) and VEGF (50 ng/ml) and the cells were cultivated for 14 days. Additional feeding of the cultures was carried out depending on the proliferation of the cells. In this case, the supernatant was removed carefully with a pipette and replaced by fresh medium. The proliferating cells contained in the supernatant were counted, adjusted to a cell density of 2×10⁶ cells/ml and introduced into fresh wells of the well plate.

Colony Assays:

Freshly isolated AC133⁺ cells and cells which had been expanded for 8 and 14 days were introduced with a cell density of 1×10³ to 5×10⁴ into a semisolid medium which consisted of 0.9% methylcellulose in IMDM, 30% FCS, 1% calf serum albumin, 10⁻⁴ mol/l mercaptoethanol and 2 mmol/l L-glutamine (complete medium from Cell Systems, St. Katharinen). In parallel batches, the cultures were stimulated either with a combination of hematopoietic growth factors consisting of SCF (50 ng/ml), IL-3 (20 ng/ml), IL-6 (20 ng/ml), G-CSF (20 ng/ml), GM-CSF (20 ng/ml) and erythropoetin (3 U/ml; complete medium from Cell Systems) or with a combination of SCGF (100 ng/ml, TEBU) and VEGF (50 ng/ml, TEBU). All cultures were carried out in quadruplicate, incubated at 37° C. in 5% CO₂ and evaluated after 14 days under the inversion microscope.

Immune Staining:

Freshly isolated AC133⁺ cells and cultivated cells were centrifuged in a cytocentrifuge at 500 rpm for 5 minutes on slides. The cytospins were air dried for at least 24 hours and then stained by immunofluorescence. The following primary non-conjugated and conjugated antibodies were used: anti-KDR-mAb (Sigma), anti-Ulex Europaeus agglutinin-1 mAb, anti-EN4 (cell systems), anti-CD31-PE (Pharmingen, Hamburg), VE-cadherin-PE (Pharmingen) and anti-vWF-FITC. Anti-mouse FITC-conjugated immunoglobulins were used as secondary antibodies. The cytospins were first washed in 10% FCS/PBS in order to block non-specific binding sites. Subsequently, the cytospins were incubated for 60 minutes at room temperature with the primary antibody. The cytospins which were incubated with a non-conjugated primary antibody were subsequently incubated for 30 minutes at room temperature. Subsequently, the cytospins were fixed with 5% glacial acetic acid/ethanol at −20° C. for 15 minutes.

Flow Cytometry:

The freshly isolated AC133+ cells were first incubated with a hemolytic buffer (0.155 mol/l NH₄Cl, 0.012 mol/l NaHCO₃, 0.1 mmol/l EDTA, pH 7.2) in order to lyse the erythrocytes. Cells which had already been cultivated were passed directly to antibody incubation. 5×10⁵ cells were incubated in each case with the following antibodies: PE-anti-AC133 mAb, FITC-anti-CD34 mAb, PE-anti-CD33 mAb, FITC-anti-CD105 mAb, PE-anti-CD14 mAb, FITC-anti-CD45 mAb, PE-anti-VE-cadherin mAb, FITC-anti-vWF mAb, PE-anti-CD31 mAb, PE-anti-c-kit mAb, FITC-anti-CD90 mAb and PE-anti-CD7 mAb. All incubations were carried out for 15 minutes at 4° C. Subsequently, the cells were washed in 0.1% BSA/PBS. The measurements were carried out as single colour and two colour analyses on a FACS SCAN flow cytometer (Becton Dickinson) and with the Cell Quest software program. Each analysis included at least 5000 counting events. An isotype control (γ1γ2a, Parmingen) was carried out concurrently with each measurement.

Synthesis of cDNA and Reverse Transcription Polymerase Chain Reaction (RT-PCR):

Freshly isolated AC133⁺ cells and cultivated cells were washed twice in PBS and centrifuged for 5 minutes at 1200 rpm and room temperature. The isolation of the RNA was carried out by means of a mini-column (Rneasy Kit, Quiagen, Hilden) in line with the manufacturer's instruction. One microgram of the isolated RNA was used for the cDNA synthesis. The cDNA synthesis was carried out using the avian myeloblastosis virus (AMV) reverse transcriptase and oligo dT as primer. Different aliquots of the cDNA were amplified by means of specific primers for KDR, Tie-2/Tek, VE-cadherin and vWF and for actin as positive control. For KDR, Tie-2/Tek, Ve-cadherin and vWF, two passages, and for actin one passage was carried out with 35 cycles of the PCR in a programmable thermoblock at 94° C. for 1.5 minutes, at 60° C. for 3 minutes and at 72° C. for 4 minutes. The PCR products were separated on 1% agarose gel, stained with ethidium bromide and visualised under UV light. The primer sequences were as follows: outer KDR sense primer 5′-GTCAAGGGAAAGACTACGTTGG-3′, outer KDR antisense primer 5′-AGCAGTCCAGCATGGTCTG-3′, inner KDR sense primer 5′-CAGCTTCCAAGTGGCTAAGG-3′, inner KDR antisense primer 5′-TCAAAAATTGTTTCTGGGGC-3′, outer Tie-2/Tek sense primer 5′-TGGACCTGTGAGACGTTC-3′, outer Tie-2/Tek antisense primer 5′-CTCTAAATTTGACCTGGCAACC-3′, inner Tie-2/Tek sense primer 5′-AGGCCAACAGCACAGTCAG-3′, inner Tie-2/Tek antisense primer 5′-GAATGTCACTAAGGGTCCAAGG-3′, outer VE-cadherin sense primer 5′-DAYCATTGGATACTCCATCCG-3′, outer VE-cadherin antisense primer 5′-ATGACCACGGGDAYGAAGTG-3′, inner VE-cadherin sense primer 5′-TTCCGAGTCACAAAAAAGGG-3′, inner VE-cadherin antisense primer 5′-TATCGTGATTATCCGTGAGGG-3′, outer vWF sense primer 5′-CTGCAAGGTCAATGAGAGAGAGG-3′, outer vWF antisense primer 5′-GAGAGCAGCAGGAGCACTG-3′, inner vWF sense primer 5′-TGAGGAGCCTGAGTGCAAC-3′, inner vWF antisense primer 5′-TGGAGTACATGGCTTTGCTG′. The specific primers for KDR, Tie-2/Tek, VE-cadherin, vWF and actin recognise encoding sequences. The size of the PCR products was as follows: for the outer KDR primer pair 591 bp, for the inner KDR primer pair 213 bp, for the outer Tie-2/Tek primer pair 624 bp, for the inner Tie-2/Tek primer pair 323 bp, for the outer VE-cadherin primer pair 462 bp, for the inner VE-cadherin primer pair 340 bp, for the outer vWF primer pair 312 bp, for the inner vWF primer pair 128 bp. In order to avoid cross-contamination, the individual operating steps of the PCR reaction and gel electrophoresis were carried out in different rooms using different pipettes. Accordingly, control reactions carried out simultaneously were always negative.

Results of Example 1:

Characterisation of the Freshly Isolated AC133⁺ Cell Population

The flow cytometric analyses gave a degree of purity of 99.94%. The total population of the AC133+cells coexpressed the surface antigens CD34, CD45, CD33 and CD31. 42.3% of the AC133⁺ cells coexpressed CD90 (thy-1), a surface marker which is expressed only on very non-mature stem cells. CD7 and c-kit, also markers for non-mature stem cells, were expressed by 15.23% and 6.86% of the AC133⁺ cells. The endothelial cell markers vWF and VE-cadherin were detectable only on 1.43% and 0.36% of the cells.

Proliferation and Differentiation of the AC133+ Cells in Suspension Culture:

Initially, the AC133⁺ cells were expanded for 14 days under the influence of Flt3 ligand, SCGF and VEGF. The cells were adherent after only a few hours following the beginning of the culture. During the first four culture days, the cells formed a monolayer of small round cells. The cell density increased substantially every day. On day 5 of the culture period, a non-adherent cell layer of small round cells was then obtained which had formed above the adherent cell layer. The non-adherent cell layer was carefully removed with a pipette, counted and introduced into fresh wells of the well plate. It was then possible to repeat the process, the cells proliferated continuously. On day 14 of the culture period, a 100 fold multiplication of the cells was achieved. The morphology changed only slightly during the entire period. On day 14, the cells had a larger diameter and exhibited a “cobblestone” morphology. On day 14, the cells were transferred into a medium which contained the growth factors SCGF and VEGF. Within three to four days, the proliferation subsided substantially and the cells exhibited the first morphological differentiation characteristics typical of endothelial cells. Initially, small elongate cells were obtained which grew while remaining very flat. After a 14 day culture in the differentiation medium, the cell population consisted predominantly of large spindle-shaped cells with a typical endothelial cell morphology.

Clonogenic Potential of the Freshly Isolated AC133⁺ Cells and the Cells in Culture:

The freshly isolated AC133⁺ cells and cells expanded for 14 days were, introduced into a semi-solid medium which contained either hematopoietic growth factors for purposes of the stimulation of hematopoietic colonies or the cytokines SCGF and VEGF for purposes of the induction of endothelial colonies. As shown in Table 1, the cells which had been expanded for 14 days in suspension cultures still exhibited a clonogenic potential. In comparison with freshly isolated AC133⁺ cells, these cells were no longer capable of forming BFU-E and CFU-E but instead had a higher capacity for forming endothelial colonies.

TABLE 1
Clonogenic potential of the freshly isolated AC133+ cells
and of the cultivated cells.

| | BFU-E | CFU-E | CFU-GEMM | CFU-GM | CFU-G | CFU-M | CFU-EC |
|---|---|---|---|---|---|---|---|
| AC133+ | 17 | 77 | 0 | 252 | 104 | 20 | 4 |
| day 0 | | | | | | | |
| Expand. | 0 | 0 | 0 | 36.5 | 6 | 24 | 33 |
| day 14 | | | | | | | |

Abbreviations: BFU-E = burst-forming unit erythrocyte; CFU-E = colony-forming unit erythrocyte; CFU-GEMM = colony-forming unit granulocyte-erythrocyte-macrophage-megakaryocyte; CFU-GM = colony-forming unit granulocyte-macrophage; CFU-G = colony-forming unit granulocyte; CFU-M = colony-forming unit macrophage; CFU-EC = colony-forming unit endothelial cell.

Identification of the Endothelial Cells:

In order to identify cells of the endothelial cell lineage, the expression of the endothelial cell markers CD31, vWF, VE-cadherin, Ulex europaeus agglutinin-1, Tie-2/Tek and KDR were examined by immune staining and RT-PCR. The results are given in Table 2 and Table 3.

TABLE 2
Percentage of positive cells for CD31, vWF, VE-cadherin and
Ulex europaeus agglutinin-1 by immunofluorescence staining.

| | CD31 | vWF | VE-cadherin | Ulex |
|---|---|---|---|---|
| AC133⁺ cells | 99% | 0 | 0 | 0 |
| day 0 | | | | |
| expanded cells | 99% | 5% | 0 | 0 |
| day 14 | | | | |
| Differentiated | 99% | 99% | 99% | 99% |
| cells day 14 | | | | |

TABLE 3
Gene expression analysis of the freshly isolated AC133+ cells
and the cultivated cells by RT-PCR.

| | KDR | Tie-2/Tek | vWF | VE-cadherin |
|---|---|---|---|---|
| AC133⁺ cells | Negative | Negative | Positive | Negative |
| day 0 | | | | |
| Expanded cells | Positive | Positive | Positive | Negative |
| day 14 | | | | |
| Differentiated | Positive | Positive | Positive | Positive |
| cells day 14 | | | | |

### Example 2

In Vivo Studies with Gene Transfected AC133⁺ Cells

In this example, the AC133⁺ cells were cultivated for 4 days at a cell density of 2×10⁶ cells/ml in IMDM+10% FCS+10% horse serum+10⁻⁶ mol/l hydrocortisone+Flt3 ligand (50 ng/ml)+SCGF (100 ng/ml)+VEGF (50 ng/ml). On day 5, 6 and 7 of the culture period, the AC133+ cells were transfected with the retroviral vector SF11αEGFPrev which encodes the enhanced green fluorescent protein. For this purpose, 6-well plates were first coated with the recombinant fibronectin fragment CH296 (RN, compare e.g. R. Kapur et al., Blood 97 (2001) 1975-81; Takara, Otsu, Japan). Subsequently, 2.5 ml/well of the retroviral supernatant previously obtained in cultures of the cell lineage PG13 were centrifuged for 30 minutes at 1000 g and 4° C. onto the RN-coated well plates. In order to load the well plates with retroviral particles to the maximum, the centrifuging process was repeated four times. Then, the AC133⁺ cells were introduced into the RN-coated well plates loaded with virus particles and incubated overnight at a cell density of 2×10⁶ cells/ml in the culture medium described above. The transfection process was repeated on the following 4 consecutive days. For this purpose, a fresh 6-well plate was coated with RN at the beginning of transfection in each case and loaded with retroviral particles in 5 centrifuging steps, as described above, and the transfection was carried out overnight. In this way, a transduction efficiency of 70% was achieved. The transfected cells were then cultivated in 6-well plates coated with fresh fibronectin, which had not been loaded with viral particles, for a further 48 hours in the above-mentioned medium under the influence of Flt3 ligand, SCGF and VEGF. On day 9 of the culture period, the cells were trypsinised, washed, resuspended in 100 μl of PBS/1×10⁶ cells and subcutaneously injected into SCID mice. In this connection, three different test groups of ten mice each were formed. In group I, a suspension of 1×10⁶ gene transfected cells plus 1×10⁶ cells of the lung carcinoma cell line A549 was injected into each mouse. The test animals of group II received 1×10⁶ gene transfected cells exclusively whereas in group III, 1×10⁶ A549 cells were applied subcutaneously exclusively. Four weeks after the injection, the tumour size and structure in all the test animals were analysed.

Subcutaneous tumours were present in all mice of group I and group III, whereas none of the animals in group II had formed a subcutaneous tumour. The largest tumours were detectable in the mice of group I. In this case, the tumour diameter was on average 30% greater than that of the tumours in group III. In frozen section preparations, the tumours of group I moreover exhibited a greater vascular density than those of group III. By fluorescence microscopy, the content of EGFP-expressing cells of the tumours was examined. Green fluorescent cells could be detected in the vessels in the case of tumours of group I.

### Example 3

In Vitro Differentiation of Liver Cells from AC133⁺ Cells

In this example, the AC133⁺ cells were cultivated at a cell density of 2×10⁶ cells/ml in Williams medium E+10% FCS+10% horse serum+5×10⁻⁶ mol/l hydrocortisone+Flt3 ligand (50 ng/ml)+SCF (100 ng/ml)+HGF (50 ng/ml)+TGF-β (10 ng/ml) in collagen-coated well plates. After 14 days, the cells were trypsinised and immunocytochemically analysed. 70% of the cells were positive for the hepatocytic marker OCH1E5 (DAKO). 20% expressed the biliar cell marker cytokeratin-19 (CK-19 (DAKO).

### Example 4

In Vitro Differentiation of Neuronal Cells from AC133⁺ Cells

In this example, the AC133⁺ cells were cultivated at a cell density of 2×10⁶ cells/ml in DMEM/F-12 (1:1)+5×10⁻³ mol/l hepes buffer+0.6% glucose+3×10⁻³ mol/l sodium bicarbonate+2×10⁻³ mol/l glutamine+25 μg/ml insulin+100 μg/ml transferrin+50 ng/ml BDNF+50 ng/ml GDNF+EGF (20 ng/ml)+bFGF (20 ng/ml) in uncoated well plates. After 14 days, the cells were analysed immunocytochemically. 62% of the cells were positive for the marker glial fibrillary acidic protein (GFAP; Incstar, Stillwater, Minn., USA), 7% of the cells expressed the microtubule-associated protein-2 (MAP-2; Boehringer Mannheim) and 3% of the cells were positive for the oligodendrocyte marker O4 (Boehringer Mannheim).

### Example 5

Stent Coating with Gene Transfected Endothelial Progenitor Cells

In this example, the AC133⁺ cells were cultivated for 4 days at a cell density of 2×10⁶ cells/ml in IMDM+10% FCS+10% horse serum+10⁻⁶ mol/l hydrocortisone+Flt3 ligand (50 ng/ml)+SCGF (100 ng/ml)+VEGF (50 ng/ml) and subsequently gene transfected, as described above, with retroviral vector SF11αEGFPrev. Following renewed culture in IMDM, Flt3 ligand, SCGF and VEGF, the cells were trypsinised on day 9 of the culture period, washed in PBS and taken up again in culture medium. In parallel, PTFE stents were coated for 2 hours with fibronectin. The coated stents were then transferred into a centrifuge tube and a layer of 3 ml of the cell-containing culture medium was placed on top. The tubes thus prepared were then centrifuged for 2 hours at 12×g and 37° C. Subsequently, the coated stents were carefully transferred into a 25 cm² culture flask with 10 ml of the above-mentioned culture medium and analysed at defined moments under the inversion fluorescence microscope. After 1 week, a confluent coating with fluorescent cells was still detectable on the stent.

### Example 6

Coating of Biomechanical Vascular Valves of the Heart with Gene Tranfected Endothelial Progenitor Cells

In a manner analogous to the above-mentioned example, the AC133⁺ cells were first cultivated 4 days at a cell density of 2×10⁶ cells/ml in IMDM+10% FCS+10% horse serum+10⁻⁶ mol/l hydrocortisone+Flt3 ligand (50 ng/ml)+SCGF (100 ng/ml)+VEGF (50 ng/ml) and subsequently gene transfected with retroviral vector SF11αEGFPrev, as described above. The cells were then cultivated again in IMDM, Flt3 ligand, SCGF and VEGF and from day 15 of the culture period onwards in IMDM, SCGF and VEGF. On day 28 of the culture period, the cells were trypsinised, washed in PBS and again taken up in culture medium. In parallel, vascular valves were coated for 2 hours with fibronectin and then introduced into a 75 cm² culture flask. In the horizontal position, 250 ml of the cell-containing culture medium were pipetted into the culture flask such that the vascular valve was completely surrounded by medium. The culture flasks were then rotated slowly for 24 hours on an automatic mixer. The automatic mixer was placed in an incubator and the culture flasks were incubated at 37° C. and 5% CO₂. Subsequently, the culture flasks were removed from the mixer, half of the culture medium was removed with a pipette and replaced by fresh medium. The coating of the vascular valve was analysed at defined moments under the inversion fluorescent microscope. In this example, too, a confluent layer of fluorescent cells was still detectable after 1 week.

### Example 7

Preparation of Blood Vessels from Endothelial Progenitor Cells In Vitro

In this example, the AC133⁺ cells were cultivated for 14 days at a cell density of 2×10⁶ cells/ml in IMDM+10% FCS+10% horse serum+10⁻⁶ mol/l hydrocortisone+Flt3 ligand (50 ng/ml)+SCGF (100 ng/ml)+VEGF (50 ng/ml) and subsequently for a further 4 days under the influence of SCGF and VEGF. The cells were then trypsinised, washed in PBS and again taken up in culture medium. In parallel, a piece of bovine pericardial tissue, 2×1 cm in length, which had been crosslinked with collagen, decellularised and photofixed (CardioFix™, Sulzer Medica, Zurich, Switzerland), was prepared and formed into a cylinder. These cylinders were then transferred into a centrifuge tube and a layer of 3 ml of the cell-containing culture medium was placed on top. bFGF (10 ng/ml) was also added to the culture medium which already contained SCGF and VEGF. The tubes prepared in this way were then centrifuged for 6 hours at 12 g and 37° C. Subsequently, the coated CardioFix cylinders were carefully transferred into a 25 cm² culture flask with 10 ml IMDM+10% FCS+10% horse serum+10⁻⁶ mol/l hydrocortisone+VEGF (50 ng/ml)+bFGF (10 ng/ml)+IGF-1 (10 ng/ml) and cultivated for 8 weeks. The cylinder was then analysed immunohistochemically. A confluent monolayer of cells with an endothelial cell morphology, which were immunohistochemically positive for vWF and VE-cadherin, was detected.

### Example 8

Diagnosis of Metastases by Means of Radioactively Labelled Endothelial Progenitor Cells

In this example, the AC133+cells were cultivated for 14 days at a cell density of 2×10⁶ cells/ml in IMDM+10% FCS+10% horse serum+10⁻⁶ mol/l hydrocortisone+Flt3 ligand (50 ng/ml)+SCGF (100 ng/ml)+VEGF (50 ng/ml) and subsequently for a further 4 days under the influence of SCGF and VEGF. The cells were trypsinised, washed in PBS and again taken up in culture medium. In this case, the medium was supplemented with 20 U/ml of heparin and the cells were incubated for 15 minutes at room temperature. To the medium, 50 MBq of 18F-fluorodeoxyglucose were added and the cells were incubated for a further 30 minutes at 37° C. The labelling reaction was then stopped by adding PBS. The cells were centrifuged for 10 minutes at 450 g, washed twice in PBS and resuspended in 100 μl/2×10⁸ cells of 0.9% NaCl solution. Subsequently, the radioactively labelled cells were injected into the tail vein of tumour carrying naked rats. Each animal received 2×10⁸ labelled cells. Measuring of the animals took place after 30, 60, 90 and 120 minutes by positron emission tomography. In this case, a maximum build-up of the labelled cells in the tumour tissue was found after 90 minutes.

Abbreviations Used:

SCGF stem cell growth factor
SCF stem cell factor
VEGF Vascular endothelial growth factor; according to the invention, the use of the isoforms A, B, C and/or D is included.
EGF epidermal growth factor
aFGF acidic fibroblast growth factor
bFGF basic fibroblast growth factor
IGF-1 insulin-like growth factor-1
NGF nerve growth factor
TGF-β1 transforming growth factor-β1
Flt3 ligand fetal liver tyrosine kinase 3 ligand
G-CSF granulocyte colony-stimulating factor
GM-CSF granulocyte-macrophage colony-stimulating factor
M-CSF macrophage colony-stimulating factor
CTNF ciliary neurotrophic factor
KGF keratinocyte growth factor
IL-1, -3, -6, -11 Interleukin-1, -3, -6, -11
PlGF placenta-like growth factor
TNFα tumor necrosis factor α
PD-ECGF platelet-derived endothelial-cell growth factor
TPO thrombopoietin
EPO erythropoietin
AP-1, -2 angiopoietin-1, -2
LIF leukemia inhibiting factor
ECGS endothelial cell growth supplement
CEACAM CEA-related cell adhesion molecule
HGF hepatocyte growth factor
GDNF glial cell line-derived neurotrophic factor
BDNF brain-derived neurotrophic factor
PDGF-BB platelet-derived growth factor-bb
BMP-4 bone morphogenetic protein-4
IMDM Iscove's modified Dulbecco's medium
MEM minimum essential medium
RPMI RPMI 164b medium
EGM-2 endothelial growth medium-2
GFP green fluorescent protein
DMSO dimethyl sulfoxide
HSA Human serum albumin
FBS Fetal calf serum

### REFERENCES

SCGF Hiraoka et al., Proc. Natl. Acad. Sci. USA 94, 7577-7582, 1997
SCF Rottapel et al., Mol. Cell. Biol. 11, 3043-3051, 1991
VEGF Houck et al., Mol. Endocrinol., 5, 1806-1814, 1991
EGF Mc Clure et al., J. Cell. Physiol. 107, 195-207, 1981
aFGF Wang et al., Mol. Cell. Biol. 9, 2387-2395, 1989
bFGF Maciag et al., Proc. Natl. Acad. Sci. USA 76, 5674-5678, 1979
Pleiotrophin Merenmies J, Rauvala H, J. Biol. Chem. 265, 1621-1624, 1990;
   Tesuka et al., Biochem. Biophys. Res. Commun. 173, 246-251, 1990;
   Li et al., Science 250, 1690-1694, 1990
PlGF Maglione et al., Proc. Natl. Acad. Sci. USA 88, 9267-9271, 1991
TGFα Lee et al., Nature 313, 489-491, 1985
TNFα Shirai et al., Nature 313, 803-806, 1985;
   Wang et al., Science 228, 149-154, 1985;
   Marmenout et al., Eur J. Biochem. 152, 515-522, 1985
Angiogenin Kurachi et al., Biochemistry 24, 5494-5499, 1985
PD-ECGF Miyazono K, Heldin C H, Biochemistry 28, 1704-1710, 1989;
   Hagiwara et al., Mol. Cell Biol. 11, 2125-2132, 1991
Proliferin Parfett et al., Mol. Cell Biol. 5, 3289-3292, 1985;
   Fassett et al., Endocrinology 141, 1863-1871, 2000
IL-1 Mc Kernan et al., Cell Immunol. 80, 84-96, 1983
IL-3 Yang et al., Cell 47, 3-10, 1986
IL-6 Fiers et al., Immunol. Lett. 16, 219-226, 1987
IL-11 Paul et al., Proc. Natl. Acad. Sci. USA 87, 7512-7519, 1990
TPO Lok et al., Nature 369, 565-568, 1994
   Bartley et al., Cell 77, 1117-1124, 1994
   de Sauvage et al., Nature 369, 533-538, 1994
   Sohma et al., FEBS Lett. 353, 57-61, 1994
EPO Gasson et al., Nature 315, 768-771, 1985
AP-1 Davis et al., Cell 87, 1161-1169, 1996
AP-2 Kim et al., J. Biol. Chem. 275, 18550-1856, 2000
LIF Gearing et al., EMBO J. 6, 3995-4002, 1987
CEACAM Ergün et al., Mol. Cell 5, 311-320, 2000
HGF Nakamura et al., Nature 342, 440-443, 1989
GDNF Lin et al., J. Neurochem. 63, 758-768, 1994
   Suranto et al., Hum. Mol. Genet. 6, 1267-1273, 1997
BDNF Leibrock et al., Nature 341, 149-152, 1989
PDGF-BB Cook et al., Biochem. J. 281, 57-65, 1992
BMP-4 Kurihara et al., Biochem. Biophys. Res. Comm. 192, 1049-1056, 1993
Neutrophin Hohn et al., Nature 344, 339-341, 1990
CNTF Negro et al., Eur. J. Biochem. 201, 289-294, 1991
Anti-CD7 mAb Foa et al., Cell Immunol. 89, 194-201, 1984
Anti-CD31 mAb Newman P J et al., Science 247, 1219-1222, 1990
Anti-CD34 mAb Sovalat et al., Hematol. Cell Ther. 40, 259-268, 1998
Anti-CD45 mAb Hall L R et al., J. Immunol. 141, 2781-2787, 1988
Anti-CD54 mAb Maio et al., Blood 76, 783-790, 1990
Anti-CD90 mAb Palmer et al., Clin. Exp. immunol. 59, 529-538, 1985
Anti-CD114 mAb Toba et al., Cytometry 35, 249-259, 1999
Anti-CD116 mAb Toba et al., Cytometry 35, 249-259, 1999
Anti-CD117 mAb Ricotti et al., Blood 91, 2397-2405, 1998
Anti-CDw123 mAb Toba et al., Cytometry 35, 249-259, 1999
Anti-CD127 mAb de Waele et al., Eur. J. Hematol. 66, 178-187, 2001
Anti-CD133 mAb Miraglia et al., Blood 90, 5013-5021, 1997
Anti-CD135 mAb Rappold et al., Blood 90, 111-125, 1997
Anti-CD140b mAb Foss et al., Eur. J. Hematol. 66, 365-376, 2001
Anti-CD144 mAb Joop et al., Thromb. Hemost. 85, 810-820, 2001
Anti-CD164 mAb Zannatino et al., Blood 92, 2613-2828, 1998
Anti-CD172a mAb Seiffert et al., Blood 94, 3633-3643, 1999
Anti-CD173 mAb Cao et al., Glycobiology 11, 677-683, 2001
Anti-CD174 mAb Cao et al., Glycobiology 11, 677-683, 2001
Anti-CD175 mAb Doyle et al., Hum. Genet. 33, 131-146, 1976
Anti-CD176 mAb Karsten et al., Hybridoma 14, 37-44, 1995
Anti-CD184 mAb Tanaka et al., J. Virol. 75, 11534-11543, 2001
Anti-CD201 mAb Tsuneyoshi et al., Thromb. Hemost. 85, 356-361, 2001
Anti-CD202b mAb William et al., Circ. res. 87, 370-377, 2000
Anti-CD224 mAb Petersen et al., Hepatology 27, 433-445, 1998
Anti-CD227 mAb Brossart et al., Cancer Res. 61, 6846-6850, 2001
Anti-CD228 mAb Johnson et al., Hybridoma 1, 381-385, 1982
Anti-CD243 mAb Del Poeta et al., Leuk. Res. 23, 451-465, 1999
Anti-EGF-R mAb Ramos-Suzarte et al., J. Nucl. Med. 40, 768-775, 1999
Anti-FGF-R2 mAb Wu et al., Acta Obstet. Gynecol. Scand. 80, 497-504, 2001
Anti-P1H12 mAb Solovey et al., N. Engl. J. Med. 337, 1584-1590, 1997
Anti-KDR mAb Hunt et al., Curr. Opin. Mol. Ther. 3, 418-424, 2001
   Michel et al., Hear Res. 155, 175-180. 2001
Anti-EN4 mAb Burgio et al., Clin. Exp. Immunol. 96, 170-176, 1994
Anti-BENE mAb de Marco et al., J. Biol. Chem. 276, 23009-23017, 2001
Gentransfection by Wahlers et al., Gene Ther. 8, 477-486, 2001 means of vectors
Genetransfection Gubin et al., Biotechniques 27, 1162-1164, 1999 by means of liposomes
Gene transfection Cui et al., Gene Ther. 8, 1508-1513, 2001 by means of naked DANN
Gene transfection Ear et al., J. Immunol. Methods 257, 41-49, 2001 by means of electroporation

1. Method for carrying out in vitro expansion of multipotent stem cells characterised in that multipotent stem cells are cultivated in the presence of Flt3 ligand and at least one growth factor from the group consisting of SCF, SCGF, VEGF, bFGF, insulin, NGF and TGF-β.

2. Method according to claim 1 characterised in that the following growth factors are used:
a) Flt3 ligand and VEGF,
b) Flt3 ligand, SCGF and VEGF,
c) Flt3 ligand and EGF,
d) Flt3 ligand, EGF and bFGF.

3. Method according to claim 1 or 2 characterised in that IGF-1 and/or EGF is/are additionally used.

4. Method according to claims 1 to 3 characterised in that multipotent stem cells are gene transfected during the expansion.

5. Method for the in vitro expansion and differentiation of multipotent stem cells characterised in that
a) in a first phase, a method according to claims 1 to 4 is carried out for the expansion of multipotent stem cells and
b) the expanded cells are differentiated in a second phase, the cells being
(i) cultivated for hematopoeitic differentiation in the presence of G-CSF, GM-CSF, M-CSF, IL-3, IL-6, IL-11, TPO and/or EPO,
(ii) cultivated for endothelial differentiation in the presence of VEGF, aFGF, bFGF, ECGS, AP-1, AP-2, NGF, CEACAM, pleiotrophin, angiogenin, PlGF, and/or HGF,
(iii) cultivated for mesenchymal differentiation in the presence of PDGF-BB, TGF-β and/or BMP-4,
(iv) cultivated for neuronal differentiation in the presence of NGF, CNTF, GDNF and/or BDNF or
(v) cultivated for hepatocytic differentiation in the presence of HGF.

6. Process according to claim 5 characterised in that
(i) IL-1, SCF and/or SCGF is/are also added for hematopoietic differentiation,
(ii) LIF, EGF, IGF-1, PDGF, PDECGF, TGFα, TGFβ, TNFα, estrogen, proliferin, IL-3, G-CSF, GM-CSF, EPO SCF and/or SCGF is/are also added for endothelial differentiation,
(iii) EGF, aFGF, bFGF, IGF-1, SCF and/or SCGF is/are also added for mesenchymal differentiation,
(iv) EGF, bFGF, GF-1, IL-1b, Il-6, Il-11, LIF, Flt3 ligand, SCF and/or BMP-4 is/are also added for neuronal differentiation or
(v) EGF, IGF-1, insulin, HCG, KGF, TNF-α, Flt3 ligand, SCF and/or SCGF is/are also added for hepatocytic differentiation.

7. Process according to claim 6 characterised in that, in the second phase
(i) a combination of SCF, IL-3, IL-6, G-SCF, GM-CSF and EPO is used for hematopoietic differentiation,
(ii) a combination of SCGF and VEGF is used for endothelial differentiation,
(iii) a combination of EGF, PDGF-BB, IGF-1, bFGF and BMP-4 is used for mesenchymal differentiation,
(iv) a combination of BDNF, GDNF, EGF and bFGF is used for neuronal differentiation or
(v) a combination of Flt3 ligand, SCF, HGF and TGF-β is used for hepatocytic differentiation.

8. Method for producing a pharmaceutical preparation characterised in that a method according to claims 1 to 4 is carried out and the multipotent stem cells obtained are processed for administration.

9. Method for preparing a pharmaceutical preparation characterised in that a method according to claims 5 to 7 is carried out, progenitor cells and/or matured cells being isolated and the cells obtained being processed for administration.

10. Method for preparing a pharmaceutical preparation characterised in that
a) a method according to claims 1 to 4 is carried out and
b) a method according to claims 5 to 7 is carried out in which either progenitor cells and/or matured cells are isolated,
the cells obtained in a) and b) being mixed and processed for administration.

11. Method according to claims 8 to 10 characterised in that the cells are radioactively labelled.

12. Method according to claims 8 to 11 characterised in that the cells are taken up in 0.9% saline solution.

13. Method for coating implantable materials in which a method according to claims 5 to 7 is carried out with endothelial differentiation of the cells, the material to be implanted being introduced during and/or at the end of the differentiation phase into the cell-containing culture medium in which the differentiation of the cells takes place.

14. Method according to claim 13 characterised in that the implantable materials are coated with fibronectin.

15. Method according to claim 13 or 14 characterised in that the implantable materials are coronary stents or vascular valves.

16. Use of the cells obtained according to the method of claims 1 to 7 for producing artificial tissues.

17. Use according to claim 16 characterised in that the tissue is brain, liver, kidney, heart, cartilage, bone, retinal, muscle or connective tissue or skin.

18. Method for producing artificial tissue characterised in that a method according to claims 5 to 7 is carried out and that the expanded cells are brought into contact, before or during the differentiation, with a matrix to which the cells can attach.

19. Process according to claim 18 characterised in that the method is carried out with endothelial or mesenchymal differentiation and the matrix is brought into contact, during the differentiation phase, with endothelial or mesenchymal progenitor cells.

20. Process according to claim 18 or 19 characterised in that the tissue is brain, liver, kidney, heart, cartilage, bone, retinal, muscle or connective tissue or skin.

21. Process for producing artificial blood vessels characterised in that a method according to claim 18 is carried out with endothelial differentiation and the cells are brought into contact with a cylindrical matrix.

22. Pharmaceutical composition obtained by a method according to claims 8 to 12.

23. Implantable material obtained by a method according to claim 13 or 14.

24. Implantable material according to claim 23 characterised in that it is a coronary stent or a vascular valve.

25. Artificial tissue which is produced by using the cells obtained by a method of claims 1 to 7.

26. Artificial tissue which is obtained by a method according to claim 18 or 19.

27. Artificial tissue according to claim 25 or 26 characterised in that it is brain, liver, kidney, heart, cartilage, retinal, muscle or connective tissue or skin.

28. Artificial blood vessel obtained by a method according to claim 21.
